SEQUENCE LISTING (10 sequences)

SEQ ID NO: 1 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 2 - DNA, 2403 bp, Homo sapiens
tttgcaggtgcctcaggaaccctgaagctgggctgagccatgatgctgctgccagaacccctgcagagggcctggtttcaggagactcagagtcctctgtgaaaaagcccttggagagcgccccagcagggctgcacttggctcctgtgaggaaggggctcaggggtctgggcccctccgcctgggccgggctgggagccaggcgggcggctgggctgcagcaatggaccgtgagctggcccagcccgcgtccgtgctgagcctgcctgtcgtctgtggccatgcccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctatgccctggggctggtgagtggagggagtgcccaagagtcccaggggaacacgggcctcccagacgtggagcgccatgagctcaagggagtgtgcccagagccccctggcctagatgaccccctggcccaggtccttaatggagcaggagtgtcctgatgattcatggagtttgccccttcctaagggaaggagatctttatctttctggttggcttgaccagtcacgttgggagaagagagagagtgccaggagaccctgagggcagccggttcctactttggactgagagaagggagccccaggctggagcagcatgaggcccagcaagaagggcttgggttctgaggaagcagatgtttcatgctgtgaggccttgcaccaggtgggggccacagcaccagcagcatctttgctgggcaggcccagccctccactgcagaagcatctggaagcaccaccttgtctccacagagcagcttgggcacagcagactggcctggccctgagactggggagtggctccaatagcctcctgccacccacacaccactctccctagactctcctagggttcaggagctgctgggcccagaggtgacatttgacttttttccaggaaaaatgtaagtgtgaggaaaccctttttattttattacctttcactctctggctgctgggtctgccgtcggtcctgctgctaacctggcaccagagcctctgcccggggagcctcaggcagtcctctcctgctgtcacagctgccatccacttctcagtcccagggccatctcttggagtgacaaagctgggatcaaggatagggagttgtaacagagcagtgccagagcatgggcccaggtcccaggggagaggttggggctggcaggccactggcatgtgctgagtagcgcagagctacccagtgagaggccttgtctaactgcctttccttctaaagggaatgtttttttctgagataaaataaaaacgagccacatcgtgttttaagcttgtccaaatgaaaaaaaaaaaaaaaaa

SEQ ID NO: 3 - DNA, 18 bp, Artificial
  primer
aaggagttgtttccgtta

SEQ ID NO: 4 - DNA, 18 bp, Artificial
  primer
aagggttacttgggttgc

SEQ ID NO: 5 - DNA, 23 bp, Artificial
  primer
ggtccttagccactccttctgtg

SEQ ID NO: 6 - DNA, 24 bp, Artificial
  primer
gatgctaccaaactggatataatc

SEQ ID NO: 7 - DNA, 25 bp, Artificial
  primer
atggcccctcccacctgccggctcc

SEQ ID NO: 8 - DNA, 28 bp, Artificial
  primer
tcagttagccttgcctttgttcagtatc

SEQ ID NO: 9 - DNA, 20 bp, Artificial
  primer
gtaacccgttgaaccccatt

SEQ ID NO: 10 - DNA, 20 bp, Artificial
  primer
ccatccaatcggtagtagcg

(19) United States
(12) Patent Application Publication
(10) Pub. No.: US 2009/0041751 A1
(43) Pub. Date: Feb. 12, 2009

(54) Sepsis Prevention Through Adenosine Receptor Modulation

(75) Inventors: Gyorgy Hasko, Scotch Plains, NJ (US); Zoltan Nemeth, East Orange, NJ (US); David Bleich, Westfield, NJ (US); Edwin Deitch, Short Hills, NJ (US)

Correspondence Address: FOX ROTHSCHILD LLP, P O BOX 592, 112 NASSAU STREET, PRINCETON, NJ, 08542-0592, US

(73) Assignee: UNIVERSITY OF MEDICINE AND DENTISTRY OF NEW JERSEY, NEW BRUNSWICK, NJ (US)

(21) Appl. No.: 11/815,276

(22) PCT Filed: Feb. 1, 2006
(86) PCT No.: PCT/US2006/003523

§ 371 Date: Aug. 1, 2007

Related U.S. Application Data
(60) Provisional application No. 60/648,809, filed on Feb. 1, 2005.

(51) Int. Cl. A61K 39/395; A61K 38/16; A61K 31/7105; A61K 31/53; A61K 31/56; A61K 38/28; A61P 31/02

(52) U.S. Cl. 424/130.1; 514/12; 514/44; 514/245; 514/171; 514/4

(57) ABSTRACT

Methods for treating sepsis or septic shock in a patient comprising administering to said patient a therapeutically effective amount of a composition containing an adenosine A₂ₐ receptor antagonist.

### CROSS-REFERENCE TO RELATED APPLICATIONS

This application claims priority under 35 U.S.C. § 119(e) to U.S. Provisional Application Ser. No. 60/648,809, which was filed on Feb. 1, 2005. The disclosure of this application is incorporated herein by reference.

### BACKGROUND OF THE INVENTION

Sepsis is the single greatest cause of non-cardiac death in the hospital setting. Approximately 800,000 episodes of sepsis occur throughout the United States alone leading to more than 200,000 deaths annually. Sepsis is a complex systemic syndrome that involves infection, inflammation, and ultimately multi-organ system failure.

At present, there are limited therapeutic options for improving patient outcome in sepsis. Current therapeutic options include antibiotics, fluids, vasopressors, supportive intensive care, and, occasionally, low-dose corticosteroids. New therapeutic agents like activated protein C provide marginal benefit to select patients with sepsis.

Adenosine receptors play an important role in modulating the innate immune response. Adenosine and inosine are potent endogenous anti-inflammatory and immunosuppressive molecules that are released from cells into the extracellular space at sites of inflammation and tissue injury. Once released, adenosine and inosine diffuse to the cell membrane of surrounding cells and bind specific cell-surface receptors. The four known adenosine receptors are G-protein coupled receptors. The genes for these receptors have been analyzed in detail and are designated A₁, A₂ₐ, A_{2b}, and A₃. Each adenosine receptor has its unique signal transduction mechanism, ligand affinity, and tissue distribution.

Conventional thinking was that the high levels of adenosine generated during sepsis caused decreased morbidity and mortality. Since high levels of adenosine were believed to dampen the excess inflammatory response during sepsis, attempts were made to accentuate the effect of adenosine on its cognate receptor through the use of adenosine receptor agonists. However, these experiments were conducted using an inadequate model of sepsis that relied on injecting lipopolysaccharide (endotoxin) into mice. In this model adenosine receptor agonists protected mice against endotoxin-induced death. However, clinical trials in human beings that relied on this model and used inhibitors of endotoxin action failed to provide any benefit (See U.S. Pat. Nos. 6,740,655 and 6,605,592)

Therefore, a need exists for more reliable therapeutic methods for treating sepsis in a patient.

### SUMMARY OF THE INVENTION

This need is met by the present invention. The present invention provides a new way to treat sepsis or septic shock by modulating adenosine receptors. The present invention is based upon the discovery that modulation of an adenosine receptor subtype decreases mortality and prevents organ dysfunction in murine septic shock induced by the cecal ligation and puncture technique. The utility of adenosine receptor modulation in protecting against septic shock using a pharmacologic adenosine receptor modulator is demonstrated.

Therefore, it is an object of the present invention to identify compounds useful for treating sepsis or septic shock in a patient by assaying a library of compounds for adenosine receptor A₂ₐ antagonist activity. Library compounds include small molecules, antibodies, peptides, small interfering RNAs, antisense RNAs, and the like.

There is also provided, in accordance with another aspect of the present invention, a method for treating sepsis or septic shock in a patient by administering a therapeutically effective amount of a composition containing an adenosine A₂ₐ receptor antagonist to the patient. Any form of adenosine A₂ₐ receptor blockade can be utilized to prevent or improve the outcome in sepsis. The adenosine A₂ₐ receptor inhibitor may include, but is not limited, to pharmacological agents that impair receptor function, small molecules, antibodies that block the receptor, peptides or proteins that block or inhibit the receptor, small interfering RNA molecules that impair or inhibit transcription of a gene encoding the adenosine A₂ₐ receptor, anti-sense RNA that impairs or inhibits the transcription of a gene encoding the adenosine A₂ₐ receptor, agents that lead to inhibition, down-regulation, or interference with adenosine A₂ₐ receptor activity, or ribozymes with a complementary base pair binding portion that binds to adenosine A₂ₐ receptor mRNA and a catalytic portion that cleaves said mRNA.

Furthermore, adenosine A₂ₐ, receptor inhibition may work synergistically with other agents previously used for the treatment of sepsis in human beings. For example, other agents known to treat sepsis include antibiotics, corticosteroids, activated protein C, and insulin.

This invention uses a target receptor to test a library of adenosine receptor modulators for protection against sepsis. This discovery enables the screening of libraries of small molecules, antibodies, peptides, small interfering RNAs, anti-sense RNA, and other agents that can deactivate adenosine A₂ₐ receptor signaling and, ultimately, protect against sepsis.

The adenosine A₂ₐ receptor inhibitor thus includes, but is not limited to pharmacological agents that impair receptor function (e.g. small molecules), antibodies that block the receptor, peptides or proteins that block or inhibit the receptor, small interfering RNA molecules that impair or inhibit transcription of the gene encoding the adenosine A₂ₐ receptor, anti-sense RNA that impairs or inhibits the transcription of the gene encoding adenosine A₂ₐ receptor, or other mechanisms that lead to inhibition, down-regulation, interference with the adenosine A₂ₐ receptor activity. A preferred adenosine A₂ₐ receptor inhibitor is 4-(2-[7-amino-2-(2-furyl[1,2,4]-triazolo[2,3-a[1,3,5]triazin-5-yl-aminoethyl)phenol (“ZM241385”).

Once sepsis is detected in a patient, the adenosine A₂ₐ receptor inhibitor can be administered immediately or after a delayed period of time.

Furthermore, an adenosine A₂ₐ receptor inhibitor may be co-administered with other agents previously used for the treatment of sepsis in human beings. For example, other agents for treating sepsis include antibiotics, corticosteroids, activated protein C, and insulin.

### BRIEF DESCRIPTION OF THE DRAWINGS

FIG. 1 demonstrates that A₂ₐ receptor KO mice are protected from death in septic peritonitis. A₂ₐ receptor WT and KO mice were subjected to cecal ligation and puncture (⅔ ligation and through and through puncture with a 20-gauge needle), and survival was monitored for 5 days (p<0.05, two-tailed Fisher's exact test);

FIGS. 2 A-D show the effect of A₂ₐ receptor deficiency on bacterial load in mice subjected to CLP at 16 (A and B) or 48 (C and D) hours after surgery. Dilutions of blood (A and C) or peritoneal lavage fluid (B and D) were cultured on tryptose blood agar plates, and the number of bacterial colonies was counted. Data are the mean±SEM of n=6-9 mice per group. Results are representative of at least three separate experiments. *p<0.05;

FIGS. 3 A-F illustrate the effect of A₂ₐ receptor deficiency on IL-10 (A and B), IL-6 (C and D), and MIP-2 (E and F) levels in the plasma or peritoneal lavage fluid of mice subjected to cecal ligation and puncture. Concentrations of these cytokines were measures at 16 (A, C, and E) or 48 (B, D, and F) hours after surgery. Concentrations of IL-10, MIP-2, and IL-6 were measured using ELISA. Data are the mean±SEM of n=6-9 mice per group. Results are representative of at least three separate experiments. **p<0.01;

FIGS. 4 A-H show lessened cleavage of caspase-3 and poly (ADP-ribose) polymerase (PARP) in A₂ₐ receptor KO mice. Cleaved forms of caspase-3 (A and B) and PARP (C and D) were detected using antibodies raised against the cleaved forms of these enzymes by Western blotting of thymus (A and C) and spleen (B and D) samples taken from A₂ₐ receptor WT and KO mice 16 hours after cecal ligation and puncture (CLP). Approximately equal loading of proteins is demonstrated by β-actin Western blotting (E and F). Results are representative of three separate experiments for each group. G, Average percentage of annexin V-positive thymocytes by flow cytometry. Thymocytes were isolated 16 hours after the onset of CLP-induced sepsis. Data are the mean±SEM of n=3-5 mice per group. Results are representative of three separate experiments. *p<0.05. H, Decreased DNA fragmentation in A₂ₐ receptor KO mice. DNA fragmentation was quantitated using TUNEL immunohistochemistry (light microscopy, 600×) of spleen samples obtained 16 hours after the CLP procedure.

FIGS. 5 A-C represent (A) RT-PCR analysis demonstrates that levels of IL-10, IL-6, and MIP-2 mRNA are decreased in spleens of A₂ₐ KO mice when compared to WT mice. F4/80⁺ macrophages from spleens (B) or peritoneal cavity (C) of A₂ₐ KO mice (n=5) exhibit increased MHC II expression (mean fluorescence intensity) when compared to WT (n=8) animals. Spleens or peritoneal cells were taken 16 hours after cecal ligation and puncture. *p<0.05;

FIGS. 6 A-E show treatment with the selective A₂ₐ receptor antagonist 4-(2-[7-amino-2-(2-furyl[1,2,4]-triazolo[2,3-a[1,3,5]triazin-5-yl-aminoethyl)phenol (“ZM241385”) (15 mg/kg, s.c, twice daily) at time 0 (A) or 2 hours after (B) resuscitation protects mice from death induced by cecal ligation and puncture. 4-(2-[7-amino-2-(2-furyl[1,2,4]-triazolo[2,3-a[1,3,5]triazin-5-yl-aminoethyl)phenol (“ZM241385”)- or vehicle-treated mice were subjected to cecal ligation and puncture (⅔ ligation and through and through puncture with a 20-gauge needle), and survival was monitored for 5 days (p<0.05, two-tailed Fisher's exact test). IL-10 (C) and MIP-2 (E) levels are decreased in the plasma and peritoneum of mice treated with the selective A₂ₐ receptor antagonist 4-(2-[7-amino-2-(2-furyl[1,2,4]-triazolo[2,3-a[1,3,5]triazin-5-yl-aminoethyl)phenol (“ZM241385”) (15 mg/kg, s.c., twice daily). IL-6 levels are attenuated in the peritoneum of 4-(2-[7-amino-2-(2-furyl[1,2,4]-triazolo[2,3-a[1,3,5]triazin-5-yl-aminoethyl)phenol (“ZM241385”)-treated mice as compared to vehicle-treated mice (D). Concentrations of IL-10, MIP-2, and IL-6 were measured by ELISA in plasma and peritoneal lavage fluid that were obtained 16 hours after cecal ligation and puncture. Data are the mean±SEM of n=6-9 mice per group. Results are representative of three separate experiments. *p<0.05; **p<0.01;

FIGS. 7 A-D demonstrate A₂ₐ receptor KO mice have increased circulating TNF-α (A) and IL-6 (B) levels following LPS administration when compared to WT mice. Circulating levels of IL-10 (C) and MIP-2 (D) are not different in A₂ₐ receptor WT and KO mice. Mice were injected intraperitoneally with LPS (5 mg/kg), and 4 hours later, the animals were sacrificed and blood collected. Cytokines from the plasma were detected using ELISA. Data are the mean±SEM of n=15-16 mice per group. *p<0.05;

FIG. 8 is a table listing laboratory markers in A₂ₐ KO and WT mice 0, 16, and 48 hours after cecal ligation puncture;

FIG. 9 shows that the A₂ₐ receptor antagonist 4-(2-[7-amino-2-(2-furyl[1,2,4]-triazolo[2,3-a[1,3,5]triazin-5-yl-aminoethyl)phenol (“ZM241385”) was associated with improved survival in CD-1 mice. Sepsis was induced in mice by cecal ligation and puncture (CLP). To determine the role of A₂ₐ receptors, we utilized CD-1 mice treated with the A₂ₐ receptor antagonist 4-(2-[7-amino-2-(2-furyl[1,2,4]-triazolo[2,3-a[1,3,5]triazin-5-yl-aminoethyl)phenol (“ZM241385”). Survival after CLP was recorded for 2 days. We assessed the immune status of mice by measuring cytokine levels from blood and peritoneal lavage fluid; and

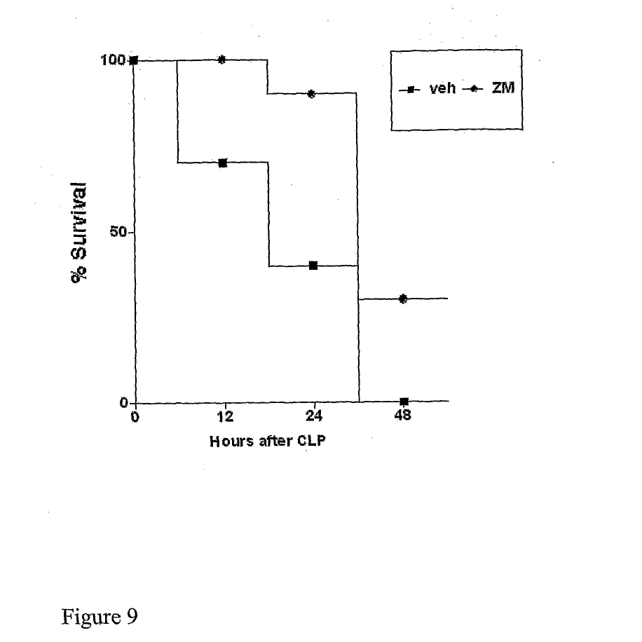

FIG. 10 demonstrates that selective adenosine A₂ₐ antagonist, ZM241358, decreases IL-10 and MIP-2 levels, but not IL-12 levels in mouse cecal-ligation and puncture sepsis model. Decreased levels of IL-10 and MIP-2 were found in 4-(2-[7-amino-2-(2-furyl[1,2,4]-triazolo[2,3-a[1,3,5]triazin-5-yl-aminoethyl)phenol (“ZM241385”) mice as compared to vehicle treated mice, while IL-12 concentrations were comparable.

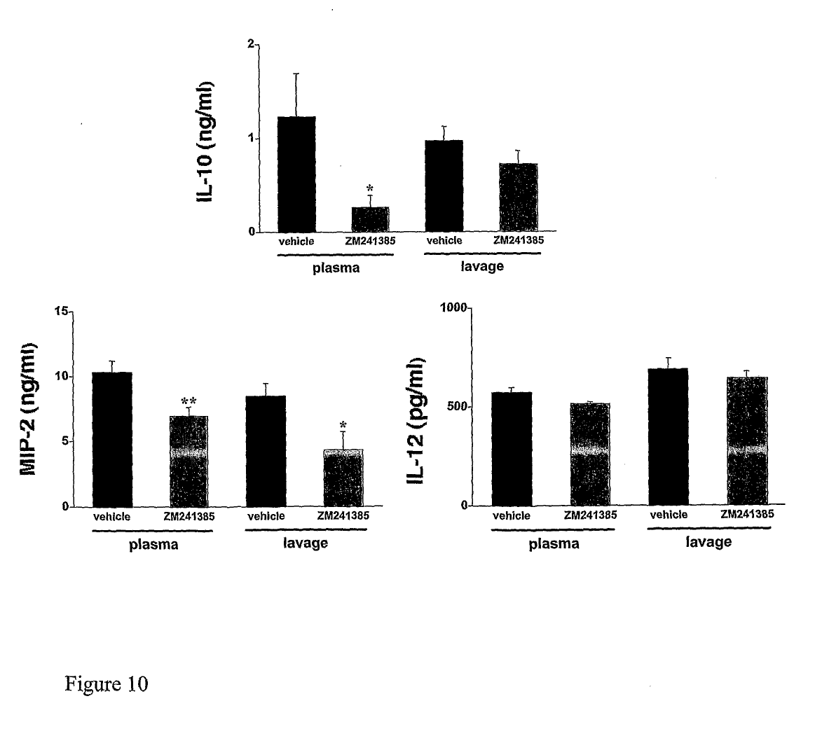

### DETAILED DESCRIPTION OF THE INVENTION

The present invention incorporates the discovery that excess adenosine receptor activation impairs immune response and survival by decreasing the inflammatory response against bacterial pathogens responsible for sepsis. The adenosine receptor A₂ₐ subtype has been identified as being responsible for the regulation of immune function and organ damage in sepsis. Thus, A₂ₐ receptor blockade is therapeutically useful for the treatment of septic shock.

### Assay Methods

There are a variety of assay methods that may be used to identify adenosine receptor A₂ₐ antagonist compounds. Representative assay methods include the in vitro and in vivo assays as disclosed in U.S. Pat. Nos. 6,916,811; 6,897,216; 6,653,315; and 6,630,475, the disclosures of all four of which are incorporated herein by reference.

### Compounds

Compounds already identified as antagonists for the A₂ₐ receptor in the above-cited patents are suitable for use in the present invention. Additional adenosine A₂ₐ receptor antagonists include, but are not limited to, those disclosed in: Chase, et al., “Translating A₂ₐ antagonist KW6002 from animal models to parkinsonian patients,” Neurology 61(11 Suppl 6):S107-11 (Dec. 9, 2003); Zocchi, et al., “The non-xanthine heterocyclic compound SCH 58261 is a new potent and selective A₂ₐ adenosine receptor antagonist,” J Pharmacol Exp Ther. 276(2):398-404 (February 1996); Kanda, et al., “KF17837: a novel selective adenosine A₂ₐ receptor antagonist with anticataleptic activity,” Eur J Pharmacol. 256(3):263-8 (May 2, 1994); Jacobson, et al, “Structure-activity relationships of 8-styrylxanthines as A2-selective adenosine antagonists,” J Med Chem. 36(10):1333-42 (May 14, 1993); Minetti, et al., “2-n-Butyl-9-methyl-8-[1,2,3]triazol-2-yl-9H-purin-6-ylamine and analogues as A₂ₐ adenosine receptor antagonists. Design, synthesis, and pharmacological characterization,” J Med Chem. 48(22):6887-96 (Nov. 3, 2005); P. Jenner, “Istradefylline, a novel adenosine A₂ₐ receptor antagonist, for the treatment of Parkinson's disease,” Expert Opin Investig Drugs. 14(6):729-38 (June 2005); Pastorin, et al, “Synthesis, biological and modeling studies of 1,3-di-n-propyl-2,4-dioxo-6-methyl-8-(substituted)1,2,3,4-tetrahydro[1,2,4]-triazolo[3,4-f]-purines as adenosine receptor antagonists,” Farmaco 60(8):643-51 (August 2005); Peng, et al. “Novel bicyclic piperazine derivatives of triazolotriazine and triazolopyrimidines as highly potent and selective adenosine A₂ₐ receptor antagonists,” J Med Chem. 47(25):6218-29 (Dec. 2, 2004); and Baraldi, et al., “7-Substituted5-amino-2-(2-furyl)pyrazolo[4,3-e]-1,2,4-triazolo[1,5-c]pyrimidines as A₂ₐ adenosine receptor antagonists: a study on the importance of modifications at the side chain on the activity and solubility,” J Med Chem. 45(1):115-26 (Jan. 3, 2002), the disclosures of all of which are incorporated herein by reference. Analogs of these compounds, which exhibit adenosine A₂ₐ receptor antagonist activity, are also suitable for use in the present invention.

The term “analog” relates to any compound which is derived from an adenosine A₂ₐ receptor antagonist and which substantially maintains the activity of the adenosine A₂ₐ receptor antagonist from which it was derived.

### Adenosine A₂ₐ Receptor Gene Expression

In another preferred embodiment, the present invention relates to a method of reducing sepsis-related damage to a cell or increasing resistance to sepsis-related damage to a cell, comprising decreasing adenosine A₂ₐ receptor activity by reducing the expression of a gene encoding the adenosine A₂ₐ receptor. This reduction in expression can be accomplished by a variety of methods and in preferred embodiments it is accomplished by altering the gene such that the gene encodes a dysfunctional or non-functional adenosine A₂ₐ receptor.

The term “expression” comprises both endogenous expression and overexpression by transduction.

A variety of means are available for altering a gene to effect expression. In a special embodiment the expression of a gene encoding the adenosine A₂ₐ receptor is reduced by contacting the gene, or an mRNA transcribed from the gene, with a compound comprising a polynucleotide selected from the group consisting of an antisense oligonucleotide, a ribozyme, a small interfering RNA (siRNA), and a short hairpin RNA (shRNA). In certain embodiments the compound comprises a polynucleotide comprising a nucleotide sequence complementary to a nucleotide sequence encoding a polypeptide comprising the amino acid sequence of SEQ ID NO: 1, (adenosine A₂ₐ receptor polypeptide sequence). In a particularly preferred embodiment the compound comprises a nucleotide sequence complementary to a nucleotide sequence comprising the nucleotide sequence of SEQ ID NO: 2 (adenosine A₂ₐ receptor polynucleotide sequence).

The term “polynucleotide” means a polynucleic acid, in single or double stranded form, and in the sense or antisense orientation, complementary polynucleic acids that hybridize to a particular polynucleic acid under stringent conditions, and polynucleotides that are homologous in at least about 60 percent of its base pairs, and more preferably 70 percent of its base pairs are in common, most preferably 90 percent, and in a special embodiment 100 percent of its base pairs. The polynucleotides include polyribonucleic acids, polydeoxyribonucleic acids, and synthetic analogues thereof. The polynucleotides are described by sequences that vary in length, that range from about 10 to about 5000 bases, preferably about 100 to about 4000 bases, more preferably about 250 to about 2500 bases. A preferred polynucleotide embodiment comprises from about 10 to about 30 bases in length. A special embodiment of polynucleotide is the polyribonucleotide of from about 10 to about 22 nucleotides, more commonly described as small interfering RNAs (siRNAs). Another special embodiment are nucleic acids with modified backcartilages such as peptide nucleic acid (PNA), polysiloxane, and 2′-O-(2-methoxy)ethylphosphorothioate, or including non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection.

The term “antisense nucleic acid” refers to an oligonucleotide that has a nucleotide sequence that interacts through base pairing with a specific complementary nucleic acid sequence involved in the expression of the target such that the expression of the gene is reduced. Preferably, the specific nucleic acid sequence involved in the expression of the gene is a genomic DNA molecule or mRNA molecule that encodes (a part of) the gene. This genomic DNA molecule can comprise regulatory regions of the gene, or the coding sequence for the mature gene.

The term ‘complementary to a nucleotide sequence’ in the context of antisense oligonucleotides and methods should be understood as sufficiently complementary to such a sequence as to allow hybridization to that sequence in a cell, i.e., under physiological conditions.

The term “hybridization” means any process by which a strand of nucleic acid binds with a complementary strand through base pairing. The term “hybridization complex” refers to a complex formed between two nucleic acid sequences by virtue of the formation of hydrogen bonds between complementary bases. A hybridization complex may be formed in solution (e.g., C0t or R0t analysis) or formed between one nucleic acid sequence present in solution and another nucleic acid sequence immobilized on a solid support (e.g., paper, membranes, filters, chips, pins or glass slides, or any other appropriate eEF2 to which cells or their nucleic acids have been fixed). The term “stringent conditions” refers to conditions that permit hybridization between polynucleotides and the claimed polynucleotides. Stringent conditions can be defined by salt concentration, the concentration of organic solvent, e.g., formamide, temperature, and other conditions well known in the art. In particular, reducing the concentration of salt, increasing the concentration of formamide, or raising the hybridization temperature can increase stringency.

### Antisense

The down regulation of gene expression using antisense nucleic acids can be achieved at the translational or transcriptional level using an expression-inhibitory agent. Antisense nucleic acids of the invention are preferably nucleic acid fragments capable of specifically hybridizing with all or part of a nucleic acid encoding an adenosine A₂ₐ receptor or the corresponding messenger gene or mRNA. In addition, antisense nucleic acids may be designed which decrease expression of the nucleic acid sequence capable of encoding an adenosine A₂ₐ receptor by inhibiting splicing of its primary transcript. Any length of antisense sequence is suitable for practice of the invention so long as it is capable of down-regulating or blocking expression of a nucleic acid coding for adenosine A₂ₐ receptor. Preferably, the antisense sequence is at least about 17 nucleotides in length. The preparation and use of antisense nucleic acids, DNA encoding antisense RNAs and the use of oligo and genetic antisense is known in the art.

The term “expression inhibitory agent” means a polynucleotide designed to interfere selectively with the transcription, translation and/or expression of a specific polypeptide or protein normally expressed within a cell. More particularly, “expression inhibitory agent” comprises a DNA or RNA molecule that contains a nucleotide sequence identical to or complementary to at least about 17 sequential nucleotides within the polyribonucleotide sequence coding for a specific polypeptide or protein. Exemplary expression inhibitory molecules include ribozymes, double stranded siRNA molecules, self-complementary single-stranded siRNA molecules, genetic antisense constructs, and synthetic RNA antisense molecules with modified stabilized backbones.

One embodiment of expression-inhibitory agent is a nucleic acid that is antisense to a nucleic acid comprising SEQ ID NO: 2. For example, an antisense nucleic acid (e.g. DNA) may be introduced into cells in vitro, or administered to a subject in vivo, as gene therapy to inhibit cellular expression of nucleic acids comprising SEQ ID NO: 2. Antisense oligonucleotides preferably comprise a sequence containing from about 17 to about 100 nucleotides and more preferably the antisense oligonucleotides comprise from about 18 to about 30 nucleotides. Antisense nucleic acids may be prepared from about 10 to about 30 contiguous nucleotides complementary to a nucleic acid sequence selected from the sequences of SEQ ID NO: 2.

The antisense nucleic acids are preferably oligonucleotides and may consist entirely of deoxyribo-nucleotides, modified deoxyribonucleotides, or some combination of both. The antisense nucleic acids can be synthetic oligonucleotides. The oligonucleotides may be chemically modified, if desired, to improve stability and/or selectivity. Since oligonucleotides are susceptible to degradation by intracellular nucleases, the modifications can include, for example, the use of a sulfur group to replace the free oxygen of the phosphodiester bond. This modification is called a phosphorothioate linkage. Phosphorothioate antisense oligonucleotides are water soluble, polyanionic, and resistant to endogenous nucleases. In addition, when a phosphorothioate antisense oligonucleotide hybridizes to its mRNA target, the RN202-315NA duplex activates the endogenous enzyme ribonuclease (RNase) H, which cleaves the mRNA component of the hybrid molecule.

In addition, antisense oligonucleotides with phosphoramidite and polyamide (peptide) linkages can be synthesized. These molecules should be very resistant to nuclease degradation. Furthermore, chemical groups can be added to the 2′ carbon of the sugar moiety and the 5 carbon (C-5) of pyrimidines to enhance stability and facilitate the binding of the antisense oligonucleotide to its TARGET site. Modifications may include 2′-deoxy, O-pentoxy, O-propoxy, O-methoxy, fluoro, methoxyethoxy phosphorothioates, modified bases, as well as other modifications known to those of skill in the art.

### Ribozyme

Another type of expression-inhibitory agent that reduces the levels of mRNA is the ribozyme. Ribozymes are catalytic RNA molecules (RNA enzymes) that have separate catalytic and substrate binding domains. The substrate binding sequence combines by nucleotide complementarity and, possibly, non-hydrogen bond interactions with its mRNA sequence. The catalytic portion cleaves the mRNA at a specific site. The substrate domain of a ribozyme can be engineered to direct it to a specified mRNA sequence. The ribozyme recognizes and then binds adenosine A₂ₐ receptor mRNA through complementary base pairing. Once it is bound to the correct adenosine A₂ₐ receptor mRNA site, the ribozyme acts enzymatically to cut the adenosine A₂ₐ receptor mRNA. Cleavage of the mRNA by a ribozyme destroys its ability to direct synthesis of the corresponding polypeptide. Once the ribozyme has cleaved its adenosine A₂ₐ receptor mRNA sequence, it is released and can repeatedly bind and cleave at other mRNAs.

Ribozyme forms include a hammerhead motif, a hairpin motif, a hepatitis delta virus, group I intron or RNaseP RNA (in association with an RNA guide sequence) motif or Neurospora VS RNA motif. Ribozymes possessing a hammerhead or hairpin structure are readily prepared since these catalytic RNA molecules can be expressed within cells from eukaryotic promoters (Chen, et al. (1992) Nucleic Acids Res. 20:4581-9). A ribozyme of the present invention can be expressed in eukaryotic cells from the appropriate DNA vector. If desired, the activity of the ribozyme may be augmented by its release from the primary transcript by a second ribozyme (Ventura, et al. (1993) Nucleic Acids Res. 21:3249-55).

The term “vectors” relates to plasmids as well as to viral vectors, such as recombinant viruses, or the nucleic acid encoding the recombinant virus.

Ribozymes may be chemically synthesized by combining an oligodeoxyribonucleotide with a ribozyme catalytic domain (20 nucleotides) flanked by sequences that hybridize to the adenosine A₂ₐ receptor mRNA after transcription. The oligodeoxyribonucleotide is amplified by using the substrate binding sequences as primers. The amplification product is cloned into a eukaryotic expression vector.

Ribozymes are expressed from transcription units inserted into DNA, RNA, or viral vectors. Transcription of the ribozyme sequences are driven from a promoter for eukaryotic RNA polymerase I (pol (I), RNA polymerase II (pol II), or RNA polymerase III (pol III). Transcripts from pol II or pol III promoters will be expressed at high levels in all cells; the levels of a given pol II promoter in a given cell type will depend on nearby gene regulatory sequences. Prokaryotic RNA polymerase promoters are also used, providing that the prokaryotic RNA polymerase enzyme is expressed in the appropriate cells (Gao and Huang, (1993) Nucleic Acids Res. 21:2867-72). It has been demonstrated that ribozymes expressed from these promoters can function in mammalian cells (Kashani-Sabet, et al. (1992) Antisense Res. Dev. 2:3-15).

### siRNA

A particularly preferred inhibitory agent is a small interfering RNA (siRNA). siRNA, preferably short hairpin RNA (shRNA), mediate the post-transcriptional process of gene silencing by double stranded RNA (dsRNA) that is homologous in sequence to the silenced RNA. siRNA according to the present invention comprises a sense strand of 17-25 nucleotides complementary or homologous to a contiguous 17-25 nucleotide sequence selected from the group of sequences encoding SEQ ID NO: 2, and an antisense strand of 17-25 nucleotides complementary to the sense strand. The most preferred siRNA comprises sense and anti-sense strands that are 100 percent complementary to each other and the adenosine A₂ₐ receptor polynucleotide sequence. Preferably the siRNA further comprises a loop region linking the sense and the antisense strand. A self-complementing single stranded siRNA molecule polynucleotide according to the present invention comprises a sense portion and an antisense portion connected by a loop region linker. The loop can be any length but is preferably 4-30 nucleotides long. Self-complementary single stranded siRNAs form hairpin loops and are more stable than ordinary dsRNA. In addition, they are more easily produced from vectors.

Analogous to antisense RNA, the siRNA can be modified to confirm resistance to nucleolytic degradation, or to enhance activity, or to enhance cellular distribution, or to enhance cellular uptake, such modifications may consist of modified internucleoside linkages, modified nucleic acid bases, modified sugars and/or chemical linkage the siRNA to one or more moieties or conjugates.

### Compositions

The present invention also provides biologically compatible, sepsis-related tissue damage-inhibiting compositions comprising an effective amount of one or more compounds identified as adenosine A₂ₐ receptor inhibitors, and/or the expression-inhibiting agents as described hereinabove. In certain aspects, the invention relates to a pharmaceutical composition for the treatment or prevention of a condition involving tissue damage associated with sepsis or a susceptibility to tissue damage associated with sepsis, comprising a therapeutically effective amount of a compound that inhibits an adenosine A₂ₐ receptor. In another aspect, the invention relates to a pharmaceutical composition for the treatment of tissue damage associated with sepsis or a susceptibility to tissue damage associated with sepsis, comprising a therapeutically effective amount of a compound comprising a polynucleotide comprising a nucleotide sequence complementary to a nucleotide sequence encoding a polypeptide comprising the amino acid sequence of SEQ ID NO: 1.

The term “effective amount” or “therapeutically effective amount” means that amount of a compound or agent that will elicit the biological or medical response of a subject that is being sought by a medical doctor or other clinician.

A biologically compatible composition is a composition, that may be solid, liquid, gel, or other form, in which the compound, polynucleotide, vector, and antibody of the invention is maintained in an active form, e.g., in a form able to effect a biological activity. For example, a compound of the invention would have inverse agonist or antagonist activity on the adenosine A₂ₐ receptor; a nucleic acid would be able to replicate, translate a message, or hybridize to a complementary mRNA of an adenosine A₂ₐ receptor; a vector would be able to transfect an adenosine A₂ₐ receptor cell and expression the antisense, antibody, ribozyme or siRNA as described hereinabove; an antibody would bind an adenosine A₂ₐ receptor polypeptide domain.

A preferred biologically compatible composition is an aqueous solution that is buffered using, e.g., Tris, phosphate, or HEPES buffer, containing salt ions. Usually the concentration of salt ions will be similar to physiological levels. Biologically compatible solutions may include stabilizing agents and preservatives. In a more preferred embodiment, the biocompatible composition is a pharmaceutically acceptable composition.

In practice, a composition containing an adenosine A₂ₐ receptor inhibitor may be administered in any variety of suitable forms, for example, by inhalation, topically, parenterally, rectally or orally; more preferably orally. More specific routes of administration include intravenous, intramuscular, subcutaneous, intraocular, intrasynovial, colonical, peritoneal, transepithelial including transdermal, ophthalmic, sublingual, buccal, dermal, ocular, nasal inhalation via insufflation, and aerosol.

Antibodies according to the invention may be delivered as a bolus only, infused over time or both administered as a bolus and infused over time. Those skilled in the art may employ different formulations for polynucleotides than for proteins. Similarly, delivery of polynucleotides or polypeptides will be specific to particular cells, conditions, locations, etc.

A composition containing an adenosine A₂ₐ receptor inhibitor may be presented in forms permitting administration by the most suitable route. The invention also relates to administering pharmaceutical compositions containing at least one adenosine A₂ₐ receptor inhibitor which are suitable for use as a medicament in a patient. These compositions may be prepared according to the customary methods, using one or more pharmaceutically acceptable adjuvants or excipients. The adjuvants comprise, inter alia, diluents, sterile aqueous media and the various non-toxic organic solvents. The compositions may be presented in the form of oral dosage forms, or injectable solutions, or suspensions.

The choice of vehicle and the content of adenosine A₂ₐ receptor inhibitor in the vehicle are generally determined in accordance with the solubility and chemical properties of the product, the particular mode of administration and the provisions to be observed in pharmaceutical practice. When aqueous suspensions are used they may contain emulsifying agents or agents which facilitate suspension. Diluents such as sucrose, ethanol, polyols such as polyethylene glycol, propylene glycol and glycerol, and chloroform or mixtures thereof may also be used. In addition, the adenosine A₂ₐ receptor inhibitor may be incorporated into sustained-release preparations and formulations.

For parenteral administration, emulsions, suspensions or solutions of the compounds according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are used. The injectable forms must be fluid to the extent that it can be easily syringed, and proper fluidity can be maintained, for example, by the use of a coating such as lecithin, by the maintenance of the required particle size in the case of dispersion and by the use of surfactants. Prolonged absorption of the injectable compositions can be brought about by use of agents delaying absorption, for example, aluminum monostearate and gelatin. The solutions of the salts of the products according to the invention are especially useful for administration by intramuscular or subcutaneous injection. Solutions of the adenosine A₂ₐ receptor inhibitor as a free base or pharmacologically acceptable salt can be prepared in water suitably mixed with a surfactant such as hydroxypropyl-cellulose. Dispersion can also be prepared in glycerol, liquid polyethylene glycols, and mixtures thereof and in oils. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation, microfiltration, and/or by various antibacterial and antifungal agents, for example, parabens, chlorobutanol, phenol, sorbic acid, thimerosal, and the like.

Sterile injectable solutions are prepared by incorporating the adenosine A₂ₐ receptor inhibitor in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

Topical administration, gels (water or alcohol based), creams or ointments containing the adenosine A₂ₐ receptor inhibitor may be used. The adenosine A₂ₐ receptor inhibitor may be also incorporated in a gel or matrix base for application in a patch, which would allow a controlled release of compound through transdermal barrier.

For administration by inhalation, the adenosine A₂ₐ receptor inhibitor may be dissolved or suspended in a suitable carrier for use in a nebulizer or a suspension or solution aerosol, or may be absorbed or adsorbed onto a suitable solid carrier for use in a dry powder inhaler.

The percentage of adenosine A₂ₐ receptor inhibitor in the compositions used in the present invention may be varied, it being necessary that it should constitute a proportion such that a suitable dosage shall be obtained. Obviously, several unit dosage forms may be administered at about the same time. A dose employed may be determined by a physician or qualified medical professional, and depends upon the desired therapeutic effect, the route of administration and the duration of the treatment, and the condition of the patient. In the adult, the doses are generally from about 0.001 to about 50, preferably about 0.001 to about 5, mg/kg body weight per day by inhalation, from about 0.01 to about 100, preferably 0.1 to 70, more especially 0.5 to 10, mg/kg body weight per day by oral administration, and from about 0.001 to about 10, preferably 0.01 to 10, mg/kg body weight per day by intravenous administration. In each particular case, the doses are determined in accordance with the factors distinctive to the patient to be treated, such as age, weight, general state of health and other characteristics which can influence the efficacy of the compound according to the invention.

The adenosine A₂ₐ receptor inhibitor used in the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the adenosine A₂ₐ receptor inhibitor may be administered 1 to 4 times per day. Of course, for other patients, it will be necessary to prescribe not more than one or two doses per day.

The following non-limiting examples set forth hereinbelow illustrate certain aspects of the invention.

### EXAMPLES

### Materials and Methods

### Experimental Animals

The A₂ₐ receptor knockout mice used in the present study were bred on a CD-1 background in a specific pathogen free facility, using founder heterozygous male and female mice. All mice were maintained in accordance with the recommendations of the “Guide for the Care and Use of Laboratory Animals”, and the experiments were approved by the New Jersey Medical School Animal Care Committee. WT and KO littermates of heterozygous parents were used exclusively in all studies. At weaning, a 0.5-cm tail sample was removed for the purpose of DNA collection for genotyping. Genotyping using RT-PCR was performed as described previously.

For pharmacological studies with the selective A_{2A} receptor antagonist 4-(2-[7-amino-2-(2-furyl)[1,2,4]triazolo[2,3-a][1,3,5]triazin-5-ylamino]ethyl)phenol(4-(2-[7-amino-2-(2-furyl[1,2,4]-triazolo[2,3-a[1,3,5]triazin-5-yl-aminoethyl)phenol (“ZM241385”); Tocris Cookson Inc.), male CD-1 mice were used that were purchased from Charles River Laboratories.

### Cecal Ligation and Puncture

Polymicrobial sepsis was induced by subjecting mice to CLP, as we have described previously, with some modifications. Six-to-eight-week-old male A₂ₐ receptor KO or WT mice were anesthetized with Nembutal (80 mg/kg), given i.p. Under aseptic conditions, a 2-cm midline laparotomy was performed to allow exposure of the cecum with adjoining intestine. Approximately two-thirds of the cecum was tightly ligated with a 3.0 silk suture, and the ligated part of the cecum perforated twice (through and through) with a 20-gauge needle (Beckton Dickinson). The cecum was then gently squeezed to extrude a small amount of feces from the perforation sites. The cecum was then returned to the peritoneal cavity and the laparotomy closed in two layers with 4.0 silk sutures. Sham-operated animals underwent the same procedure without ligation or puncture of the cecum. The mice were resuscitated with 1 ml of physiological saline injected s.c., and returned to their cages with free access to food and water. One group of mice was monitored daily and survival recorded for 10 days. Another group of mice was re-anesthetized with Nembutal (80 mg/kg; i.p.) 16 or 48 hours after the operation, and blood, peritoneal lavage fluid, and various organs were harvested as described below.

The effect of pharmacological inactivation of A_{2A} receptors in mice subjected to CLP was evaluated using CD-1 mice in a similar fashion to that described for the A_{2A} receptor KO or WT mice. In this set of experiments, the mice were injected immediately before or 2 hours after the operation and every 12 hours thereafter with 4-(2-[7-amino-2-(2-furyl[1,2,4]-triazolo[2,3-a[1,3,5]triazin-5-yl-aminoethyl)phenol (“ZM241385”) (15 mg/kg, s.c.) or its vehicle (DMSO).

### Collection of Blood, Peritoneal Lavage Fluid, and Organs

Blood samples were obtained aseptically by cardiac puncture using heparinized syringes after opening the chest and placed on ice into heparinized Eppendorf tubes until further processing for hematological and bacteriological analysis. Aliquots of whole blood were analyzed for hematology by flow cytometry (CELL-DYN 3200 System, Abbott Laboratories) in a centralized facility. After serial dilutions for bacteriological analysis were made (see below), the blood was centrifuged at 2000×g for ten minutes and the recovered plasma stored at −70° C. until further use. For peritoneal lavage, the abdominal skin was cleansed with 70% ethanol and the abdominal wall exposed by opening the skin. Four milliliters of sterile physiological saline was then installed into the peritoneal cavity via an 18-gauge needle. The abdomen was massaged gently for 1 minute while keeping the tip of the needle in the peritoneum, after which procedure peritoneal fluid was recovered through the needle. Recovered peritoneal lavage fluid was placed on ice until processed for bacteriological examination. After serially diluting the peritoneal lavage fluid to determine CFU numbers (see below), the peritoneal lavage fluid was centrifuged at 5000×g for ten minutes and the supernatant stored at −70° C. until further analysis. Samples from spleen, thymus, lung, kidneys, and liver were excised and either immediately frozen in liquid nitrogen or placed in 10% paraformaldehyde for subsequent histological analysis. Snap-frozen tissue samples were transferred to a −70° C. refrigerator until analyzed for gene expression and apoptotic markers.

Quantification of Bacterial CFUs from Peritoneal Lavage Fluid and Blood

Hundred microliters of blood or ten microliters of peritoneal lavage fluid was diluted serially in sterile physiological saline. Ten microliters of each dilution was aseptically plated and cultured on tryptose blood agar plates (Becton Dickinson) at 37° C. After 24 hours, the number of bacterial colonies was counted. Quantitative cultures are expressed as CFUs per milliliter of blood or peritoneal lavage fluid.

### Determination of Cytokine, Aspartate Aminotransferase (AST), Alanine Aminotransferase (ALT), and Blood Urea Nitrogen (BUN) Levels

Concentrations of IL-10, IL-6, IL-12 p70, TNF-α and MIP-2, in plasma or peritoneal lavage fluid were determined using commercially available ELISA kits (R&D Systems) and according to the manufacturer's instructions. The lower detection limit for all these cytokines was 10 pg/ml. Plasma concentrations of AST, ALT, and BUN were analyzed using standard laboratory procedures.

### Western Blot Analysis for Markers of Apoptosis

Samples of spleen and thymus were homogenized in a Dounce homogenizer in modified radioimmunoprecipitation assay buffer (50 mM Tris-HCl, 150 mM NaCl, 1 mM EDTA, 0.25% Na-deoxycholate, 1% Nonidet P-40, 1 μg/ml pepstatin, 1 μg/ml leupeptin, 1 mM PMSF, 1 mM Na₃VO₄). The lysates were transferred to Eppendorf tubes and centrifuged at 15,000×g for 15 minutes, and the supernatant recovered. Protein concentrations were determined using the Bio-Rad protein assay kit. A total of 30 to 40 μg of sample was separated on 8-16% Tris-glycine gel (Invitrogen) and transferred to nitrocellulose membrane. The membranes were probed with polyclonal rabbit anticleaved caspase-3 (Cell Signaling; #9661S), polyclonal rabbit anti-cleaved poly (ADPribose) polymerase (PARP, Cell Signaling, #9544S), or polyclonal goat anti-b-actin antibody (Santa Cruz Biotechnology Inc.; #sc-1615), and subsequently incubated with a secondary horseradish peroxidase-conjugated anti-rabbit or anti-goat antibody (Santa Cruz). Bands were detected using ECL Western Blotting Detection Reagent (Amersham).

### Apoptosis Detection by TUNEL

Paraffin blocks containing spleen tissue specimens were cut in 5 mm thick sections and the sections processed and stained for the detection of apoptosis using the TACSTM In Situ Apoptosis Detection Kit (TACS Klenow (DAB)) obtained from Trevigen Inc. (Gaithersburg, Md.), according to the manufacturer's instructions. When viewed under a standard light microscope, apoptotic nuclei can be clearly distinguished by brown staining. Quantification of the number of apoptotic cells was performed using Olympus IX71 microscope, as we have previously described. In total, 6600, 21440 and 29655 cells were examined in spleens of control (n=3), WT-CLP (n=6) and KO-CLP (n=6) groups, respectively. The results are expressed as the percent of TUNEL-positive cells, relative to the number of total cells counted in spleen sections.

### Flow Cytometry Determinations for Detection of Thymocyte Apoptosis and MHC II Expression on Splenic and Peritoneal Macrophages

To quantitate thymocyte apoptosis, tissue sections from thymi were gently glass ground to dissociate cells. Tissue debris was then removed from cell suspensions using a 70-mm nylon cell strainer (BD Falcon, San Diego, Calif.) and the cells washed twice and then resuspended in ice cold PBS. The degree of apoptotic cell death was quantified using a commercially available, fluorescein labeled Annexin V containing kit (Annexin V-FITC Apoptosis detection Kit I, BD Biosciences Pharmingen, San Diego, Calif.).

Thymocytes (3×105) were stained with FITC-labeled Annexin V and propidium iodide according to the manufacturer's instruction. Cells were analyzed in a centralized laboratory using a FACScan Flow Cytometer equipped with a 488 nm laser, 530/30-nm and 585/42-nm band pass filters and a 650-nm long-pass filter (BD Biosciences, San Jose, Calif.). Instrument calibration was performed daily employing Calibrite Beads (BD Biosciences, San Jose, Calif.) and also by sphero beads (Spherotech Inc., Libertville, Ill.) using target channel values for each of the assays used in the study. Data were analyzed using Cytomation Summit computer software (Cytomation, Inc., Fort Collins, Colo.).

Electronic compensation of the instrument was carried out to exclude overlapping of the two emission spectra. Cell counts in regions of doublets for annexin V positive only, propidium iodide positive only, double-positive, and double-negative were determined and compared.

MHC II expression on splenic and peritoneal macrophages was also determined using flow cytometry. Macrophages were identified using FITC-labeled anti-mouse CD11b (BD Pharmingen, San Diego, Calif.) and phycoerythin (PE)-labeled anti-mouse F4/80 antibodies (eBiosciences, San Diego, Calif.). MHC II expression was determined using anti-mouse APC-labeled MHC II antibody (eBiosciences). Cell suspensions from peritoneal lavage and spleen were added to tubes pre-loaded with the corresponding fluorescent-labeled antibodies. After gentle mixing, the tubes were kept at room temperature in the dark for 15 min. RBCs were then lysed with 2.0 ml of BD FACS Lysing Solution (BD Biosciences, San Jose, Calif.). After two washes cells were fixed in 0.3 ml of 3% formaldehyde and kept at 4° C. in the dark until acquisition. Analyses were performed using a FACScan flow cytometer and CellQuest software (Becton Dickinson, Mansfield, Mass.).

### Affymetrix GeneChip Analysis of Spleen Samples and RT-PCR

RNA isolation, cDNA synthesis, and cRNA transcription were performed, as previously described. cRNA was hybridized to Affymetrix murine microarrays, which contain probe sets for the whole mouse genome. Hybridization, scanning, and data analysis were performed at the Affymetrix Gene Chip Core Facility in the W. M. Keck Foundation Biotechnology Resource Laboratory at Yale University (technical details are available at http://info.med.yale.edu/wmkeck/affymetrix/). Differentially expressed genes were identified by the Biostatistics Resource Laboratory at the W. M. Keck Foundation by comparing data from spleens taken from CLP-induced A₂ₐ WT and KO mice 16 hours after the operation (n=3 per group). RT-PCR for IL-10, IL-6, MIP-2, and 18 S was carried out as described previously and using the following primers: IL-10-5′-AAGGAGTTGTTTCCGTTA-3′ (sense) and 5′-AAGGGTTACTTGGGTTGC-3′(antisense); IL-6-5′-GGTCCTTAGCCACTCCTTCTGTG-3′(sense) and 5′-GATGCTACCAAACTGGATATAATC-3′(antisense); MIP-2-5′-ATGGCCCCTCCCACCTGCCGGCTCC-3′ (sense) and 5′-TCAGTTAGCCTTGCCTTTGTTCAGTATC-3′ (anti-sense); and 18S-5′-GTAACCCGTTGAACCCCATT-3′ (sense) and 5′-CCATCCAATCGGTAGTAGCG-3′ (anti-sense).

### Endotoxemic Studies

Female A₂ₐ receptor WT or KO mice were injected intraperitoneally with LPS (5 mg/kg; from E. coli, serotype 055:B5, Sigma) in a volume of 0.1 ml/10 g body weight. 4 hours later, the animals were sacrificed and blood collected. Cytokines from the plasma were detected using ELISA, as described above.

### Statistical Analysis

Survival curves were analyzed using the two-tailed Fisher's exact test. Two-tailed t testing was used to compare cytokine concentrations, CFUs, and other laboratory parameters. Statistical significance was assigned to p values smaller than 0.05.

### Results

### Example 1

### Genetic A₂ₐ Receptor Deficiency Protects Against CLP-Induced Mortality

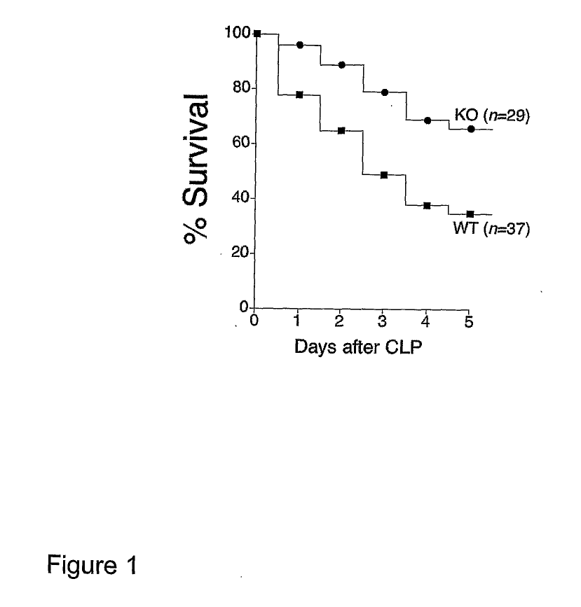

Control (WT) mice had a mortality rate of approximately 70% when recorded on day 5 after the CLP procedure (FIG. 1). This mortality rate was the result of a gradual process, which was characterized by 10-20% of the mice dying every day. No changes in mortality were detected when the mice were followed for an additional 5 days (data not shown). The mortality rate of A₂ₐ KO mice was significantly lower on each day with a ˜35% mortality rate on day 5 after CLP (FIG. 1). There were no additional deaths in this group until the termination of the experiment (10 days after the surgery, data not shown).

### Example 2

### A₂ₐ Receptor Deficiency Improves Bacterial Clearance

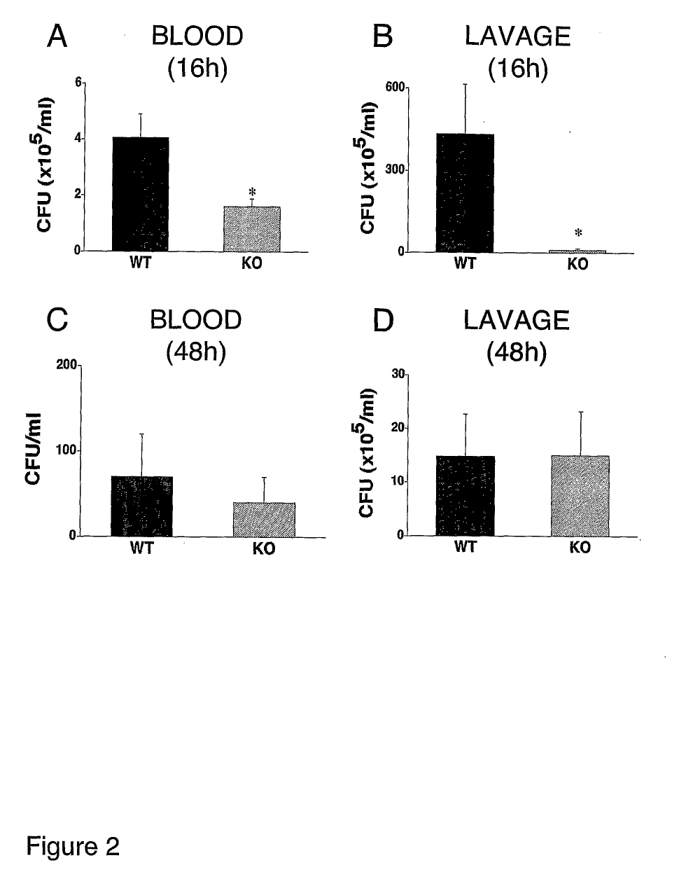

Because persistence of local bacterial infection and bloodstream invasion play important roles in mortality in the CLP model, we next assessed the impact of A₂ₐ receptor inactivation on bacterial levels at the primary peritoneal site of infection and in the blood stream. We found markedly decreased numbers of bacteria in both the blood and peritoneal lavage fluid of A₂ₐ receptor KO mice when compared to WT animals at 16 hours (FIGS. 2A and 2B). Bacterial numbers fell substantially by 48 hours after surgery in both the blood and peritoneal lavage fluid and there were no differences in CFUs between A₂ₐ KO and WT mice at this point (FIGS. 2C and 2D). Blood and peritoneal lavage fluid remained sterile in sham-operated A_{2A} receptor KO and WT mice (data not shown).

### Example 3

### Effect of Genetic A₂ₐ Receptor Inactivation on Cytokine Production and Markers of Organ Injury

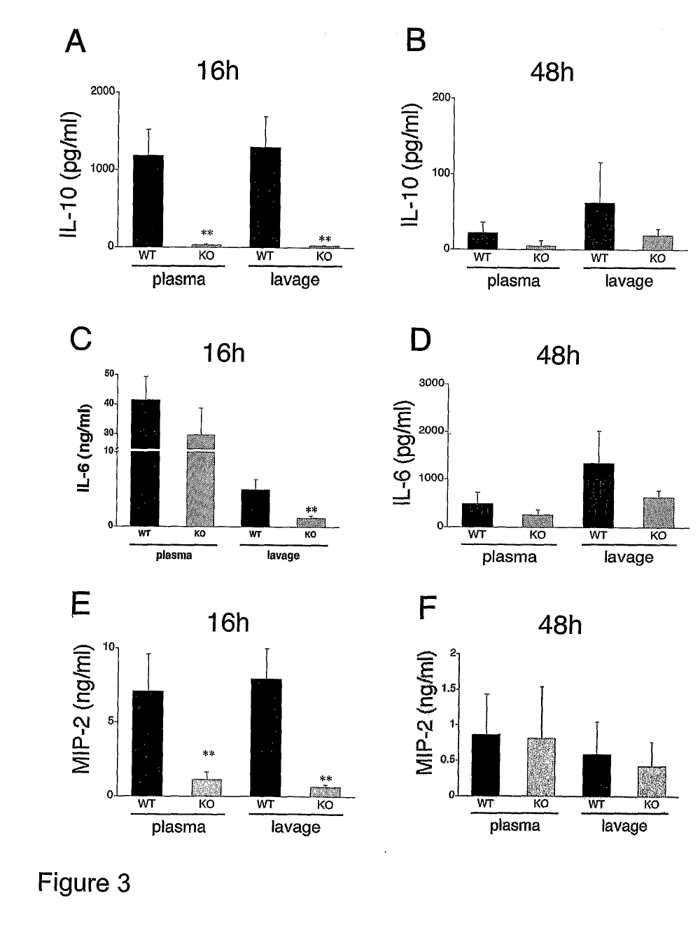

Because IL-10 appears to be an essential mediator in sepsis-induced impairment in antibacterial host defense, we compared IL-10 concentrations in the plasma and peritoneal lavage fluid obtained from A₂ₐ receptor KO and WT mice subjected to CLP or sham-operation. Sham-operated A₂ₐ receptor WT or KO mice had no detectable levels of IL-10 in their plasma or peritoneal lavage fluid (data not shown). While CLP elevated IL-10 concentrations in both the plasma and peritoneal lavage fluid in both A₂ₐ receptor KO and WT mice, A₂ₐ KO mice exhibited markedly lower levels of IL-10 at 16 hours after the CLP procedure (FIG. 3A). IL-10 concentrations subsided to comparable levels in septic A₂ₐ KO and WT mice by 48 hours (FIG. 3B).

Because IL-6 blockade with neutralizing antibodies has been shown to be protective in CLP-induced sepsis, we next explored the role of A₂ₐ receptors in regulating IL-6 production during sepsis. While IL-6 levels in sham-operated A₂ₐ receptor WT and KO mice were low and comparable between the two groups (38±20 pg/ml in the WTs versus 19±0.6 pg/ml in the KOs for the peritoneal lavage fluid and 2.53±0.01 ng/ml in the WTs versus 2.53±0.03 ng/ml in the KOs for the plasma), CLP-induced levels of IL-6 were significantly and markedly higher in the peritoneal lavage fluid but not plasma of A₂ₐ receptor WT mice than in the A₂ₐ KO animals (FIG. 3C). IL-6 concentrations decreased by 48 hours after the CLP procedure and no differences were seen in IL-6 concentrations between the A₂ₐ KO and WT mice at this point (FIG. 3D).

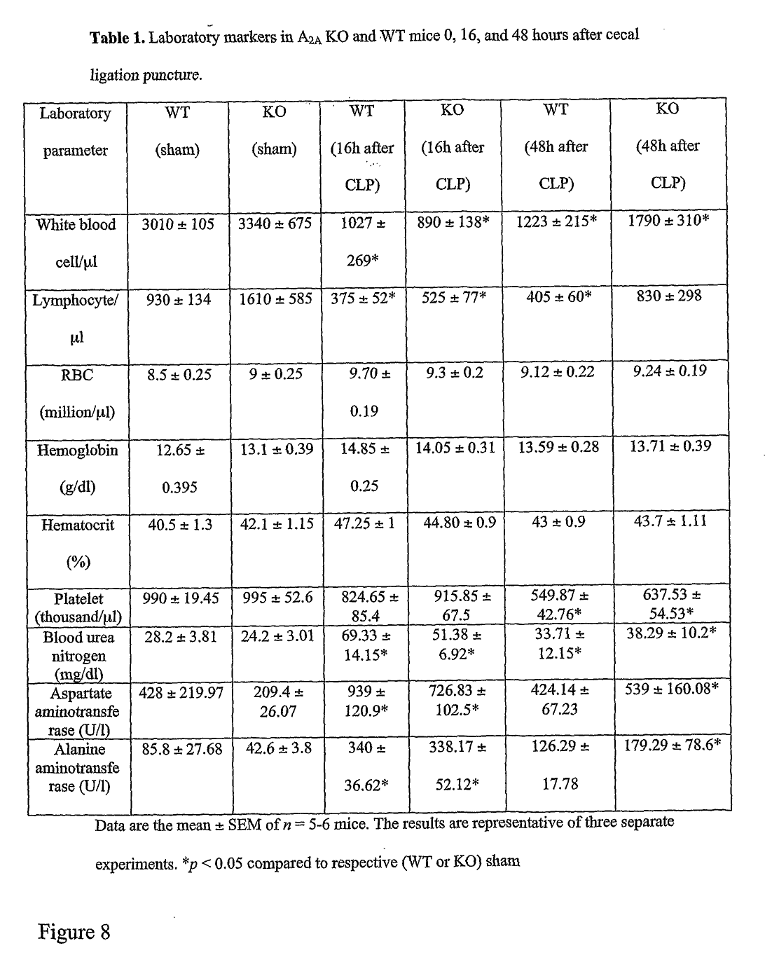

To investigate whether A₂ₐ receptor deficiency altered the formation of classical proinflammatory cytokines, we next determined concentrations of TNF-α, IL-12 p70, and MIP-2 in both the plasma and peritoneal lavage fluid. We found that the concentrations of IL-12 p70 and TNF-α were below the detection limit for our assays in all groups of mice, including sham- and CLP-operated A₂ₐ receptor WT and KO mice (data not shown). Although MIP-2 was not detectable in sham-operated WT or KO animals (data not shown), CLP-induced concentrations of MIP-2 were diminished in A₂ₐ KO mice as compared to their WT counterparts when measured at 16 hours (FIG. 3E) but not at 48 hours (FIG. 3F). CLP induced an increase in markers of kidney (BUN) and liver (AST and ALT) injury, when compared to sham-operated animals (FIG. 8). Additionally, white blood cell counts, lymphocyte numbers, and platelet counts dropped significantly in CLP-subjected mice when compared to shams (FIG. 8). However, there were no differences in the levels of these markers or hematological parameters between the WT and KO groups either at 16 (FIG. 8) or 48 hours (data not shown) after the CLP procedure.

### Example 4

### Apoptotic Markers in Lymphoid Organs of A₂ₐ Receptor KO and WT Mice Undergoing CLP

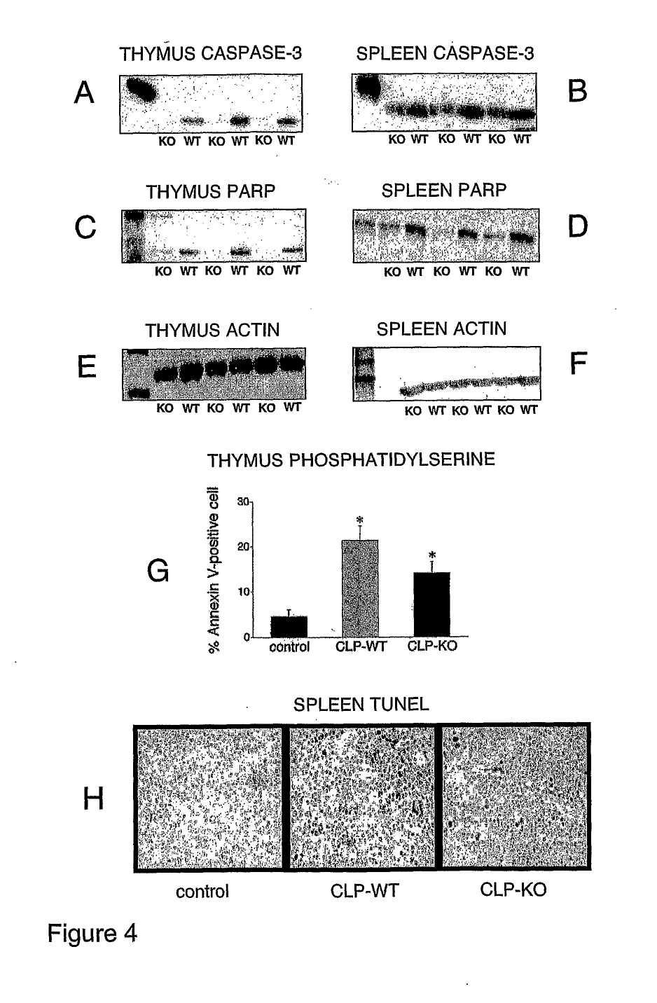

Increasing evidence shows that widespread lymphocyte depletion induced by apoptosis may contribute to the immunosuppression that occurs in sepsis. In addition, A₂ₐ receptor activation has been reported to induce lymphocyte apoptosis. Previous studies have documented that the cleavage/activation of caspase-3 is an important early indicator of apoptosis in the spleen and thymus of animals subjected to CLP-induced sepsis. PARP is a major downstream target of activated caspase-3 and is cleaved by this enzyme during apoptosis. Therefore, we tested the hypothesis that A₂ₐ receptor deficiency would prevent the cleavage of caspase-3 and PARP in the spleen and thymus of mice subjected to CLP. We found that 16 hours after the onset of sepsis, WT mice exhibited substantial cleavage of caspase-3 and PARP (FIG. 4). In contrast, the cleavage of both caspase-3 and PARP was markedly suppressed in A₂ₐ receptor KO mice (FIGS. 4A, 4B, 4C, and 4D). These indicators of apoptosis were absent in both A₂ₐ receptor WT and KO mice at 48 hours, as well as in sham-operated mice (data not shown).

Caspase-3 activation leads to the appearance of late apoptotic signs, such as phosphatidylserine exposure on the outer cell membrane. We next examined whether the decreased caspase-3 cleavage/activation in thymus of A₂ₐ KO mice translated into decreased phosphatidylserine exposure 16 hours after the onset of sepsis. Using FITC-labeled annexin V staining and flow cytometry of thymocytes, we found that CLP significantly upregulated phosphatidylserine exposure on thymocytes from both A₂ₐ receptor KO and WT animals (FIG. 4G). Although, thymocytes from KO animals exhibited 34% lower phosphatidylserine exposure than those from WT animals, this difference did not reach statistical significance (p=0.116; FIG. 4G).

Since phosphatidylserine exposure is only marginally detectable in the spleen of mice that have undergone CLP, we used TUNEL immunohistochemistry to quantify late apoptotic events in septic A₂ₐ receptor KO and WT animals. The percentage of TUNEL-positive cells in spleens of non-septic control mice was very low 0.13±0.13% (n=3; mean±SEM) (FIG. 4H). CLP significantly increased the fraction of TUNEL positive cells in WT mice to 6.59±1.32% (n=6, p<0.001;). The percentage of TUNEL-positive cells in spleens of KO mice exposed to CLP was significantly decreased as compared to WT mice from 6.59±0.54% to 4.08±0.72% (n=6, p<0.05), respectively (FIG. 4H).

### Example 5

### Splenic Gene Expression Profile in Septic A₂ₐ Receptor KO Versus WT Mice

To further assess the potential cellular and molecular mechanisms that are associated with the decreased mortality of A₂ₐ KO versus WT mice during sepsis, we compared splenic gene expression profiles in these animals. We employed oligonucleotide microarray analysis using Affymetrix chips representing the entire mouse genome. There were approximately 330 genes that were significantly up-regulated and nearly 700 genes that were down-regulated in A₂ₐ KO versus WT mice at least twofold (Supplemental data). Many of these differentially expressed genes were classified into multiple biological process categories as a result of their biological complexity (Gene Ontology-Supplemental data).

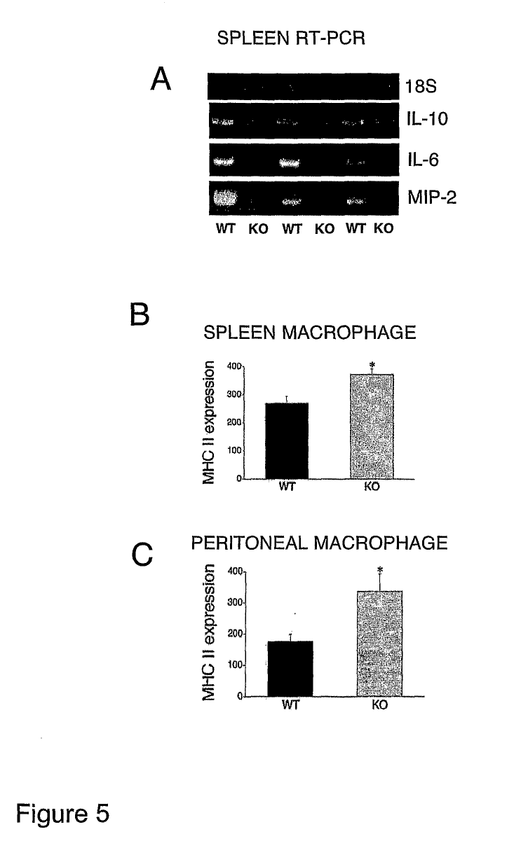

Importantly, IL-10, IL-6, and MIP-2 (chemokine (C—X—C motif) ligand 2 were among the down-regulated genes in A₂ₐ KO versus WT mice (Supplemental data). RTPCR confirmed that mRNA levels of IL-10, IL-6, and MIP-2 were decreased in spleens of A₂ₐ KO mice when compared to their WT controls (FIG. 5A). Of the up-regulated genes in A₂ₐ KO versus WT mice, the most notable differences were observed with members of the MHC II locus. In order to test whether these changes manifested at the cellular phenotypic level, we compared MHC II expression of septic KO and WT animals using flow cytometry. We found that F4/80+ splenic (FIG. 5B) and peritoneal (FIG. 5C) macrophages from septic KO animals displayed markedly elevated MHC II expression as compared to cells from WT mice. These data indicate that there are concordant decreases in the protein and mRNA levels of IL-10, IL-6, and MIP-2, as well as a concordant increase in protein and mRNA of MHC II in septic A₂ₐ KO vs. WT mice.

### Example 6

### Pharmacological Inactivation of A₂ₐ Receptors Decreases CLP-Induced Mortality

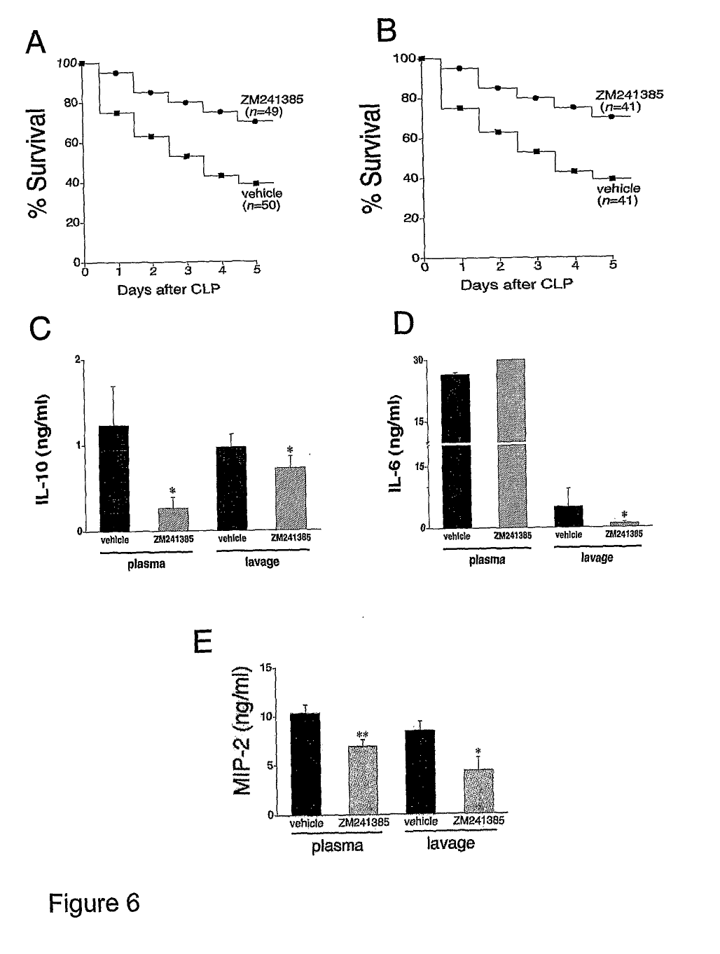

We further examined the role of A₂ₐ receptors in mediating CLP-induced mortality using a pharmacological approach. CD-1 mice treated with the selective A₂ₐ receptor antagonist 4-(2-[7-amino-2-(2-furyl[1,2,4]-triazolo[2,3-a[1,3,5]triazin-5-yl-aminoethyl)phenol (“ZM241385”) (15 mg/kg, s.c., twice daily) (49-51) starting at the time of resuscitation exhibited significantly improved survival compared to vehicle-treated mice (FIG. 6A). To explore whether this improved survival of 4-(2-[7-amino-2-(2-furyl[1,2,4]-triazolo[2,3-a[1,3,5]triazin-5-yl-aminoethyl)phenol (“ZM241385”)-treated vs. vehicle-treated mice was associated with a similar cytokine pattern to that one observed in A₂ₐ KO vs. WT mice, we measured IL-10, IL-6, and MIP-2 concentrations in the plasma and peritoneal lavage fluid at 16 hours. Levels of IL-10 and MIP-2 in both the plasma and peritoneal lavage fluid were decreased in 4-(2-[7-amino-2-(2-furyl[1,2,4]-triazolo[2,3-a[1,3,5]triazin-5-yl-aminoethyl)phenol (“ZM241385”)-treated mice as compared with vehicle-treated animals (FIGS. 6C and 6E). Similar to genetic inactivation of A₂ₐ receptors, levels of IL-6 were lower in the peritoneal fluid of 4-(2-[7-amino-2-(2-furyl[1,2,4]-triazolo[2,3-a[1,3,5]triazin-5-yl-aminoethyl)phenol (“ZM241385”)-treated mice than in the peritoneal fluid of vehicle-treated mice, however IL-6 concentrations in the plasma were comparable between the two groups (FIG. 6D).

Finally, we explored the effect of delayed administration of 4-(2-[7-amino-2-(2-furyl[1,2,4]-triazolo[2,3-a[1,3,5]triazin-5-yl-aminoethyl)phenol (“ZM241385”) relative to resuscitation. We observed that 4-(2-[7-amino-2-(2-furyl[1,2,4]-triazolo[2,3-a[1,3,5]triazin-5-yl-aminoethyl)phenol (“ZM241385”) administration starting 2 hours after resuscitation (15 mg/kg, s.c., twice daily) was still protective (FIG. 6B), indicating a potential clinical utility of A₂ₐ receptor blockade in acutely developing septic conditions.

### Example 7

### Bolus High Dose Endotoxin Increases Levels of TNF-α and IL-6 in A₂ₐ Receptor KO Mice when Compared to WT Animals

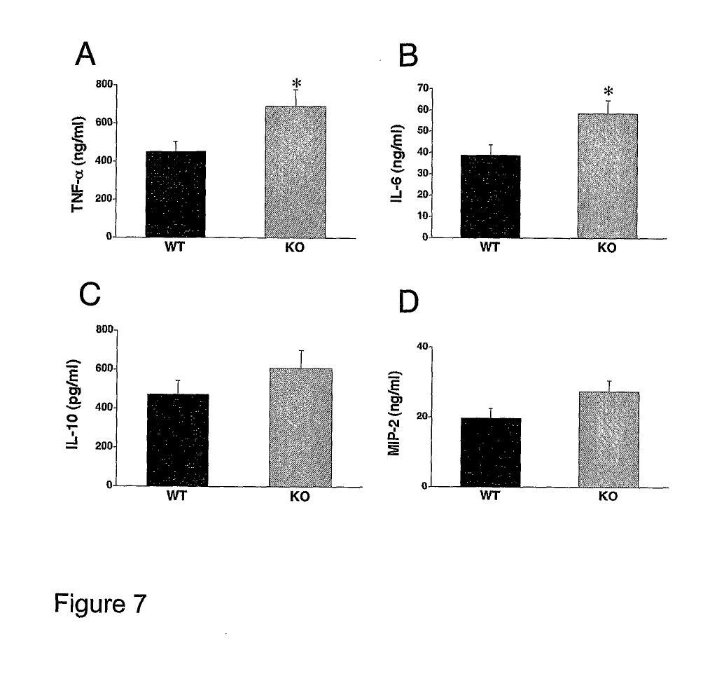

Endotoxin (LPS) treatment of mice induces an overwhelming inflammatory response with no infectious component. To investigate the role of A₂ₐ receptors in regulating this inflammatory response, we compared cytokine levels of A₂ₐ KO and WT mice injected intraperitoneally with LPS (5 mg/kg). The plasma level of both TNF-α and IL-6 was increased in A₂ₐ KO mice when compared to WT mice, whereas IL-10 and MIP-2 levels were comparable (FIG. 7). Thus, A₂ₐ receptors differentially modulate cytokine responses in sepsis and in overwhelming endotoxemia.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

What is claimed is:

1. A method for treating sepsis or septic shock in a patient comprising administering to said patient a therapeutically effective amount of a composition containing an adenosine A₂ₐ receptor inhibitor.

2. The method of claim 1, wherein the adenosine A₂ₐ receptor inhibitor is selected from the group consisting of pharmacological agents that impair receptor function, small molecules, antibodies that block the receptor, peptides or proteins that block or inhibit the receptor, small interfering RNA molecules that impair or inhibit transcription of a gene encoding the adenosine A₂ₐ receptor, anti-sense RNA that impairs or inhibits the transcription of a gene encoding the adenosine A₂ₐ receptor, agents that lead to inhibition, down-regulation, or interference with adenosine A₂ₐ receptor activity, and ribozymes with a complementary base pair binding portion that binds to adenosine A₂ₐ receptor mRNA and a catalytic portion that cleaves said mRNA.

3. The method of claim 2, wherein the adenosine A₂ₐ receptor inhibitor comprises 4-(2-[7-amino-2-(2-furyl[1,2,4]-triazolo[2,3-a[1,3,5]triazin-5-yl-aminoethyl)phenol (ZM241385).

4. The method of claim 1, wherein the composition further comprises an antibiotic, a corticosteroid, activated protein C, insulin, or a mixture thereof.

5. The method of claim 1, comprising administering the adenosine A₂ₐ receptor inhibitor immediately after sepsis is detected in said patient or following a delayed period of time after sepsis is detected in said patient.

6. A method of reducing tissue damage associated with sepsis in a mammal comprising blocking adenosine A₂ₐ receptor activity in said mammal.

7. The method of claim 6, wherein said receptor activity is blocked by contacting A₂ₐ receptors with an antagonist compound.

8. The method of claim 6, wherein said receptor activity is blocked by reducing expression of a gene encoding the receptor.

9. The method of claim 8, wherein said gene expression is reduced by contacting said gene, or an mRNA transcribed from said gene, with a compound comprising a polynucleotide selected from the group consisting of an antisense oligonucleotide, and siRNA, and an shRNA.

10. The method of claim 9, wherein said compound comprises a polynucleotide comprising a nucleotide sequence complementary to a nucleotide sequence encoding a polypeptide comprising the amino acid sequence of SEQ ID NO: 1.

11. The method of claim 9, wherein said compound comprises a nucleotide sequence complementary to a nucleotide sequence of SEQ ID NO: 2.

12. A pharmaceutical composition for treating sepsis or septic shock in a patient comprising a therapeutically effective amount of an adenosine A₂ₐ receptor inhibitor and a pharmaceutically acceptable carrier.

13. The composition of claim 12, wherein the inhibitor is selected from the group consisting of pharmacological agents that impair receptor function, small molecules, antibodies that block the receptor, peptides or proteins that block or inhibit the receptor, small interfering RNA molecules that impair or inhibit transcription of a gene encoding the adenosine A₂ₐ receptor, anti-sense RNA that impairs or inhibits the transcription of a gene encoding the adenosine A₂ₐ receptor, agents that lead to inhibition, down-regulation, or interference with adenosine A₂ₐ receptor activity, and ribozymes with a complementary base pair binding portion that binds to adenosine A₂ₐ receptor mRNA and a catalytic portion that cleaves said mRNA.

14. The composition of claim 13, wherein the inhibitor comprises 4-(2-[7-amino-2-(2-furyl[1,2,4]-triazolo[2,3-a[1,3,5]triazin-5-yl-aminoethyl)phenol (ZM241385).
